(19) United States
(12) Patent Application Publication
(10) Pub. No.: US 2011/0054426 A1
(43) Pub. Date: Mar. 3, 2011

(54) FEMALE PORTABLE URINARY COLLECTION DEVICE

(76) Inventor: Lizann STEWART, Noblesville, IN (US)

(21) Appl. No.: 12/871,663

(22) Filed: Aug. 30, 2010

Related U.S. Application Data
(60) Provisional application No. 61/238,909, filed on Sep. 1, 2009.

(51) Int. Cl. A61F 5/455

(52) U.S. Cl. 604/347

(57) ABSTRACT

A female portable urinary device including a contour funnel, a tube, and a collection pouch. One end of the tube is connected to the contour funnel and the other end of the tube is connected to the collection pouch. The contour funnel includes an outer surface that conforms to the genital area of the user.

### CROSS-REFERENCE TO RELATED APPLICATIONS

This application claims the benefit of Provisional Application No. 61/238,909 filed Sep. 1, 2009, the contents of which are incorporated herein by reference.

### BACKGROUND OF THE INVENTION

The present invention generally relates to a female portable urinary collection device that gives women the ability to urinate anywhere.

An increasing number of women are spending a greater amount of time outdoors. As a result, women are frequently left without options or very limited options when needing to urinate. Unfortunately, the complicated shape of the female genital area makes it difficult to provide a non-leaking seal between the female urinary outer part and any urinary device.

In addition, toilet seats in public restrooms are often dirty or otherwise in an unsanitary condition. Accordingly, many women are forced to bend above the toilet seat to avoid contact with the dirty seat. It is very difficult for women to bend and urinate simultaneously.

As can be seen, there is a need for a portable female urinary device that can provide a leak-free sealing engagement with the female genital area, while the female is in a standing, lying or sitting position. In addition, there is a need for a portable female urinary device for women who are partially disabled for instance in a wheel chair or elderly women that get up frequently to urinate.

### SUMMARY OF THE INVENTION

In one aspect of the present invention, the portable urinary device comprises a contour funnel; a tube having a first end connected to the contour funnel and a second end connected to a collection pouch; the contour funnel comprises an outer surface that conforms to a contour of a user genital anatomical surface.

In another aspect of the present invention, the portable urinary device comprises a contour funnel; a tube having a first end connected to the contour funnel and a second end connected to the collection pouch; a collection pouch; and a check valve connected between the second end of the tube and the collection pouch.

In another aspect of the present invention, the portable urinary device comprises a contour funnel having an outer surface that conforms to a contour of a user genital anatomical surface; a tube having a first end connected to the contour funnel and a second end connected to a collection pouch; a one-way valve connected to the second end of the tube and the collection pouch; and a liner located around an outer surface of the contour funnel.

These and other features, aspects and advantages of the present invention will become better understood with reference to the following drawings, description and claims.

### BRIEF DESCRIPTION OF THE DRAWINGS

FIG. 1 illustrates a perspective front view of the female portable urinary device according to an exemplary embodiment of the present invention;

FIG. 2 illustrates an exploded perspective view of the female portable urinary device of FIG. 2;

FIG. 3 illustrates a detailed section view of a top portion of the female portable urinary device of FIG. 1;

FIG. 4 illustrates a detailed section view of a valve, in a closed position, of the female portable urinary device of FIG. 1;

FIG. 5 illustrates a detailed section view the valve, in an open position with fluid flowing there through, of the female portable urinary device of FIG. 1; and

FIG. 6 illustrates a detailed section view of the collection pouch of the female urinary device of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a female portable urinary device that gives women the ability to urinate anytime.

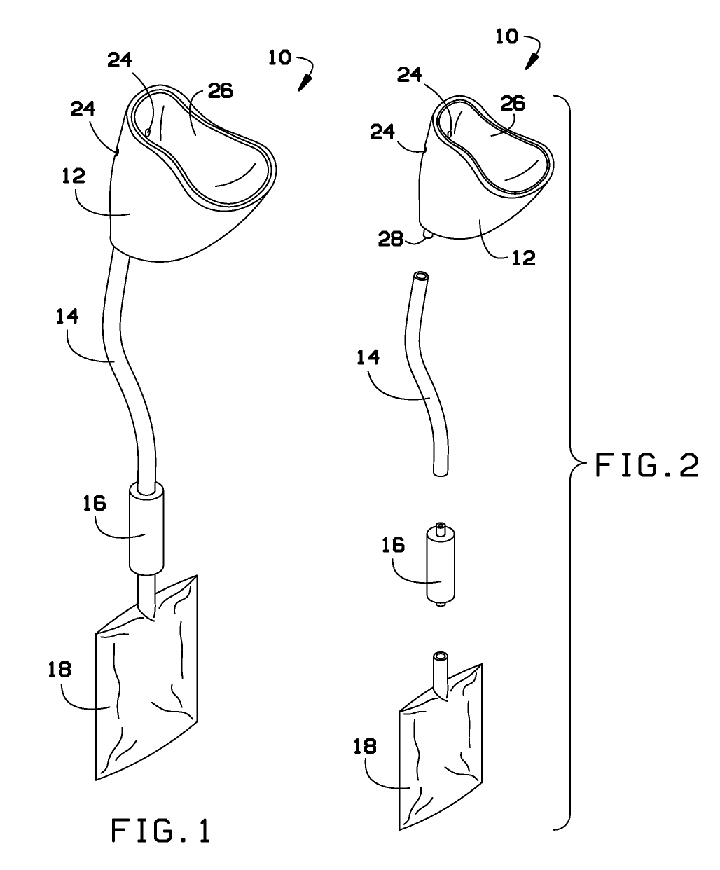

FIGS. 1-2 show one embodiment of the present invention on which the female portable urinary device 10 includes a contour funnel 12; a tube 14 connected to the contour funnel 12, and a collection pouch 18. In addition, FIG. 1 shows a valve 16 to connect and disconnect the tube 14 to the collection pouch 18.

The contour funnel 12 may be geometrically molded to conform to the genital anatomy of the user. In one embodiment, the contour funnel 12 may have the shape of the male genital anatomy. In one embodiment, the contour funnel 12 may have the shape of the female genital anatomy. The contour funnel 12 may be formed of a liquid impermeable material that provides an inexpensive and effective means for collecting the urine. In one embodiment, the contour funnel 12 may be made of a molded plastic. In one embodiment, the contour funnel 12 may be made of silicone. The contour funnel 12 may have a sealing surface that conforms to the genital-urinary area of the user. In one embodiment, the contour funnel 12 may have a pear shape. In one embodiment, one end of the contour funnel 12 may be wider than the other end of the contour funnel 12. In one embodiment, the contour funnel 12 may be a cup-shaped contour funnel having a liner 26 attached to the entire rim of the contour funnel 12. The liner 26 may be the part of the contour funnel 12 that actually touches the user's skin. The liner 26 may be made of a flexible and soft material. In one embodiment, the liner 26 may be made of silicone. The user may hold the liner 26 tightly against the genital part to form a tight seal and prevent the escape of fluid (see FIG. 6) from the contour funnel 12. In one embodiment, the fluid may be urine.

In one embodiment, at least one air vent 24 may be placed in the wall of the contour funnel 12. The air vents 24 may help create the tight seal.

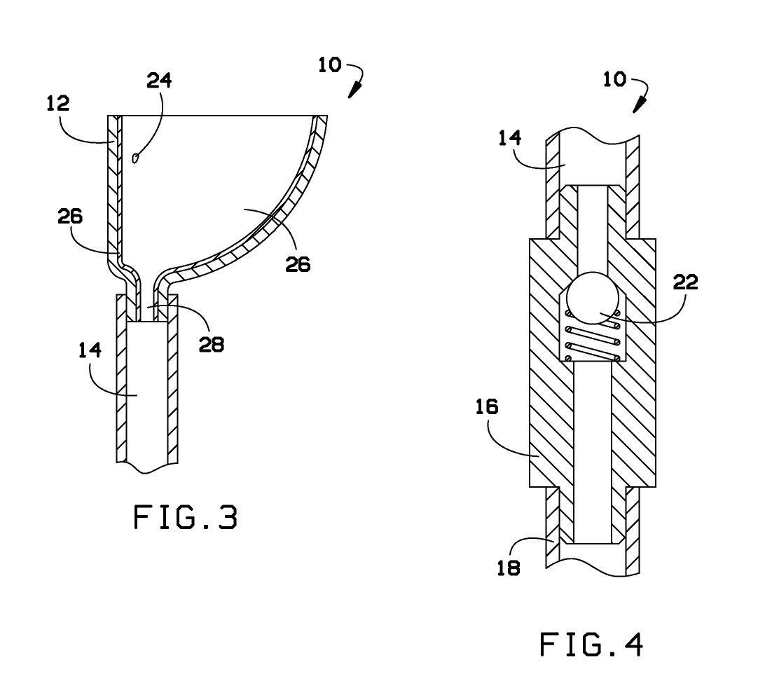

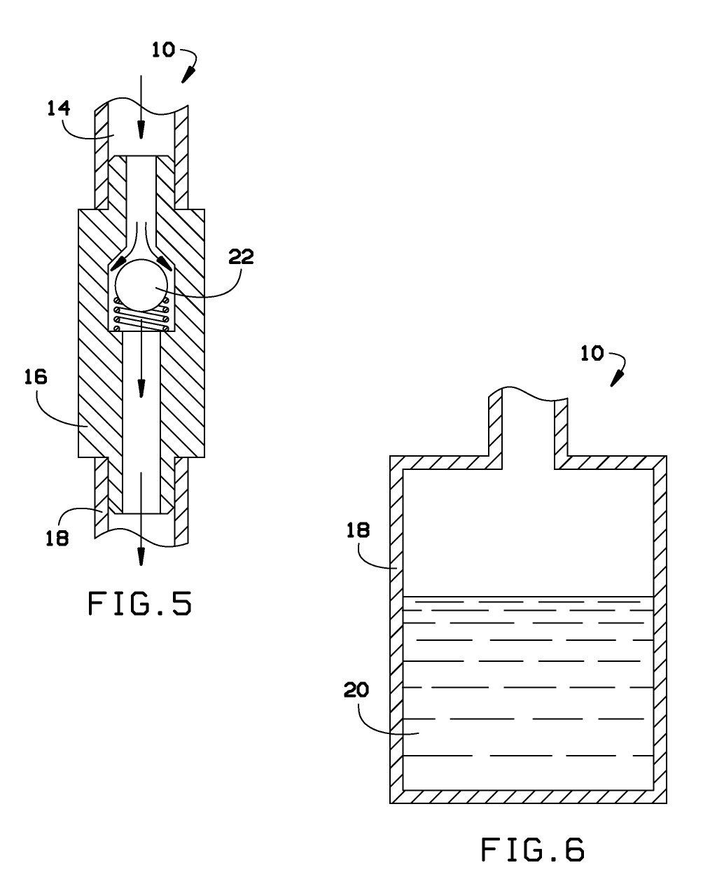

FIG. 3 shows an opening 28 located at the bottom of the contour funnel 12 to connect to the tube 14. The contour funnel 12 may collect the fluid 20 immediately as it leaves the body, funneling the fluid to the tube 14. The tube 14 may transport the fluid 20 from the contour funnel 12 to the collection pouch 18. The tube 14 may be made of a soft flexible material. The length of the tube 14 may depend on the user's requirement. In one embodiment, the length of the tube 14 may be between approximately 4 to 12 inches.

In one embodiment, the female portable urinary device 10 may not include the tube 14. In that embodiment, the contour funnel 12 may be directly connected to the valve 16.

FIG. 4 shows the valve 16 that may be located between one end of the tube 14 and the collection pouch 18. The valve 16 may prevent the fluid 20 from re-entering the tube 14. In addition, the valve 16 may prevent leakage of the fluid 20 from the collection pouch 18. In one embodiment, the valve 16 may be a check valve having a ball seal assembly 22. In one embodiment, the valve 16 may include a connector on each end. One of the connectors of the valve 16 may connect to the tube 14 or directly to the contour funnel 12. The other connector of the valve 16 may connect to the collection pouch. In one embodiment, the valve 16 may allow the fluid to flow in only one direction. In one embodiment, the valve 16 may allow the fluid to flow in both directions. On one embodiment, the connectors may be female or male connectors. In one embodiment, the valve 16 may have female connectors. In one embodiment, the valve 16 may have male connectors.

The collection pouch 18 may be made of a waterproof material. In one embodiment, the collection pouch 18 may be made of plastic or vinyl. In one embodiment, the collection pouch 18 may include a connector that interconnect with one of the connectors of the valve 16. In one embodiment, the collection pouch 18 may be removable. In one embodiment, the collection pouch 18 may include an absorbent material to solidify the urine. In one embodiment, the collection pouch 18 may have a lining to prevent leaks. In one embodiment, the collection pouch 18 may have a double lining.

In one embodiment, the portable female urinary device 10 may include a fastening device that allows the user to secure the device 10 onto the body; thus the hands of the user may be free. In one embodiment, the fastening device may be straps, belts, clips, or pins.

The user may position the contour funnel 12 over the urinary human part and a seal may be created. The user may proceed to urinate into the contour funnel 12 which will direct the fluid 20 through the opening 28 and then down the tube 14 passing through the valve 16 and then into the collection pouch 18.

After the fluid 20 has been collected into the collection pouch 18, the user may disconnect the collection pouch 18 from the tube 14.

In one embodiment, the collection pouch 18 may be reusable. In one embodiment, the collection pouch 18 may be disposable. In one embodiment, the collection pouch may have a square, round, triangular, cylindrical, or cube shape. In one embodiment, the collection pouch 18 may be a bag. In one embodiment the collection pouch may be approximately 3 inches×5 inches in a rectangular shape.

In one embodiment, the portable female urinary device 10 may not include the collection pouch 18; thus, the fluid 20 may flow freely to the toilet.

In one embodiment, the contour funnel 12 may be shaped to contour a male genital area.

The size and the shape of the contour funnel 12, the tube 14, the valve 16, and the collection pouch 18 may depend on the user's gender and age.

In one embodiment, the female portable urinary device 10 of the present invention may be used in the medical field to relieve patients before receiving a catheter. In one embodiment, the device 10 may be used by personnel in the military field.

The portable urinary device 10 of the present invention may allow the user to urinate in a standing and/or sitting position and collect the fluid without being visible to onlookers.

In one embodiment, the portable urinary device 10 of the present invention may include a carrying case to easily carry the portable urinary device 10.

It should be understood, of course, that the foregoing relates to exemplary embodiments of the invention and that modifications may be made without departing from the spirit and scope of the invention as set forth in the following claims.

We claim:

1. A portable urinary device comprising:
a contour funnel;
a tube having a first end and a second end;
a collection pouch;
wherein the first end of the tube is connected to the contour funnel;
wherein the second end of the tube is connected to the collection pouch; and
wherein the contour funnel comprises an outer surface that conforms to a contour of a user's genital anatomical surface.

2. The portable urinary device of claim 1, wherein the tube is a flexible tube.

3. The portable urinary device of claim 1, wherein the collection pouch is a disposable pouch or a reusable pouch.

4. The portable urinary device of claim 1, further comprising a liner located around an outer surface of the contour funnel.

5. A portable urinary device comprising:
a contour funnel;
a tube having a first end and a second end, wherein the first end of the tube is connected to the contour funnel;
a collection pouch, wherein the second end of the tube is connected to the collection pouch; and
a valve connected between the second end of the tube and the collection pouch.

6. The portable urinary device of claim 5, further comprising a liner located around an outer surface of the contour funnel.

7. A portable urinary device comprising:
a contour funnel, wherein the contour funnel comprises an outer surface that conforms to a contour of a user genital anatomical surface;
a tube having a first end and a second end, wherein the first end of the tube is connected to the contour funnel;
a collection pouch, wherein the second end of the tube is connected to the collection pouch;
a one-way valve connected to the second end of the tube and the collection pouch; and
a liner located around an outer surface of the contour funnel.

8. The portable urinary device of claim 7, wherein the collection pouch is a disposable collection pouch.

9. The portable urinary device of claim 7, wherein the collection pouch is a reusable collection pouch.

10. The portable urinary device of claim 7, wherein the one-way valve is a check valve.
